(19) United States
(12) Patent Application Publication
(10) Pub. No.: US 2018/0120330 A1
(43) Pub. Date: May 3, 2018

(54) PRE-SYMPTOMATIC DIAGNOSIS OF A VIRAL ILLNESS

(71) Applicant: The Johns Hopkins University, Baltimore, MD (US)

(72) Inventors: Mekbib Astatke, Gaithersburg, MD (US); Amy L. Connolly, Ijamsville, MD (US); Allyson M. Bissing-Gibson, Columbia, MD (US)

(21) Appl. No.: 15/682,723

(22) Filed: Aug. 22, 2017

Related U.S. Application Data
(60) Provisional application No. 62/416,179, filed on Nov. 2, 2016.

(51) Int. Cl. G01N 33/68

(52) U.S. Cl. CPC G01N 33/6866; G01N 2333/183; G01N 2333/57

(57) ABSTRACT

A method for a pre-symptomatic diagnosis of a viral illness includes obtaining a biological sample with a peripheral blood mononuclear cell from a subject. The method further includes stimulating the biological sample, where the stimulation of the biological sample includes adding to the biological sample a predetermined amount of antigen configured to trigger a T-cell IFN-γ response in the biological sample and a predetermined amount of reagent configured to capture the T-cell IFN-γ response. The method may even further include comparing the level of the T-cell IFN-γ response detected in the biological sample to a control sample level, the control sample level including a control sample T-cell IFN-γ response level detected in a control sample of the subject, where if the level of the T-cell IFN-γ response in the biological sample is at a predetermined amount greater than the control sample level, the subject is diagnosed with the viral illness.

### CROSS-REFERENCE TO RELATED APPLICATIONS

This application claims priority to and the benefit of prior-filed, co-pending U.S. Provisional Application Ser. No. 62/416,179 filed on Nov. 2, 2016, the entire contents of which are hereby incorporated herein by reference.

### STATEMENT OF GOVERNMENTAL INTEREST

This invention was made with Government support under contract number 2012-12-50800010 awarded by the Intelligence Advanced Research Projects Activity (IARPA). The Government has certain rights in the invention.

### TECHNICAL FIELD

This disclosure relates generally to a pre-symptomatic diagnosis of a viral illness.

### BACKGROUND

Ebola Virus Disease (EVD) is a disease that is caused by a virus of the Filoviridae family. Currently, there are four identified EVD species that are known to cause disease in humans: Zaire ebolavirus, Sudan ebolavirus, Tai Forest ebolavirus, and Bundibugyo ebolavirus. Once infected by EVD, the incubation period of EVD in a person may vary from anywhere to 2 to 21 days, with the first symptoms commonly being fever, fatigue, muscle pain, headache, and sore throat. The first symptoms are then generally followed by vomiting, diarrhea, rash, impaired kidney and liver function, and in some cases, both internal and external bleeding. Based on the initial symptoms, EVD can be difficult to clinically distinguish from other infectious diseases such as malaria, typhoid fever, and meningitis. Currently, the average EVD diagnosis is not made until five days after the onset of symptoms. The longer EVD is left untreated in a subject the more costly the treatment becomes and the greater likelihood EVD may prove fatal.

### BRIEF SUMMARY

Accordingly, in order to enable the pre-symptomatic diagnosis of viral illnesses, some example embodiments may provide a method and a diagnostic kit that are configured to detect a viral illness in a subject after exposure to the viral illness rather than upon the onset of symptoms associated with the viral illness. Accordingly, by enabling the pre-symptomatic diagnosis of viral illnesses, the treatment associated with the viral illness may be less costly and intensive, and there is a higher likelihood of recovery in the subject.

In one example embodiment, a method for a pre-symptomatic diagnosis of a viral illness in a subject is provided. The method may include obtaining a biological sample that includes at least one peripheral blood mononuclear cell from a subject. The method may further include stimulating the biological sample, where the stimulation of the biological sample includes adding to the biological sample a predetermined amount of antigen configured to trigger a T-cell IFN-γ response in the biological sample and a predetermined amount of reagent configured to capture the T-cell IFN-γ response. The method may even further include comparing the level of the T-cell IFN-γ response detected in the biological sample to a control sample level, the control sample level including a control sample T-cell IFN-γ response level detected in a control sample of the subject, where if the level of the T-cell IFN-γ response in the biological sample is at a predetermined amount greater than the control sample level, the subject is diagnosed with the viral illness.

In a further example embodiment, a diagnostic kit for a pre-symptomatic diagnosis of a viral illness in a subject is provided. The diagnostic kit may include a predetermined amount of antigen associated with the viral illness and a predetermined amount of reagent for capturing IFN-γ produced by a biological sample from the subject.

### BRIEF DESCRIPTION OF THE DRAWINGS

Having thus described example embodiments of the invention in general terms, reference will now be made to the accompanying drawings, which are not necessarily drawn to scale, and wherein:

FIG. 1 illustrates a method for a pre-symptomatic diagnosis of a viral illness according to an example embodiment;

FIG. 2 illustrates a flow diagram for a method for a pre-symptomatic diagnosis of a viral illness according to an example embodiment; and

FIG. 3 illustrates a diagnostic kit according to an example embodiment.

### DETAILED DESCRIPTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout. Furthermore, as used herein, the term “or” is to be interpreted as a logical operator that results in true whenever one or more of its operands are true.

There currently exists no technique for the diagnosis of EVD prior to the onset of clinical symptoms in a subject. In fact, EVD is not typically diagnosed until five days after the onset of symptoms of the disease. Moreover, the current methods for diagnosing EVD after the onset of symptoms are not very sensitive, resulting in a greater likelihood of a false positive or false negative result during the diagnostic testing. The accurate and early detection of EVD prior to the onset of symptoms may result in a more successful and less costly and intensive treatment plan for the subject and thus less likelihood of fatality associated with the disease. Accordingly, a method and a diagnostic kit for the pre-symptomatic diagnosis of a viral illness (e.g., EVD) is provided herein.

It should be understood that although EVD is the viral illness frequently referenced throughout, the method and diagnostic kit described herein could be applicable to a wide variety of viral illness.

As used herein, the term “antigen” is a molecule or part of a molecule that can be bound by a major histocompatibility complex (MEW) and presented to a T-cell receptor. Examples of antigens include, but are not limited to, proteins or polypeptides, peptides, polysaccharides, lipids, protozoan cell membranes, viral capsids, viral glycoproteins, foreign tissue or cells, and the body's own cells that the body fails to recognize as normal, such as infected cells.

The term “infection” refers to the invasion of a host organism's bodily tissues by disease-causing organisms, their multiplication, and the reaction of host tissues to these organisms and the toxins they produce.

The term “peripheral blood mononuclear cells (PBMC)” may include any blood cell having a round nucleus. Such cells are known to play a role in the immune response. PBMC may include for instance lymphocytes such as T-cells, B-lymphocytes and NK cells, monocytes, and macrophages. The PBMC may correspond to a B-lymphocyte. The term “B lymphocyte” may refer to B-lymphocytes at any stage of differentiation, including naive B lymphocytes, mature B-lymphocytes, memory B-lymphocytes, B1 cells, B2 cells and plasma B-lymphocytes. PBMC express markers at their cell surface, the markers differing from one PBMC population to another. For instance, B-lymphocytes express CD19 at their cell surface, helper T-cells express CD4 at their cell surface, cytotoxic T-lymphocytes express CD8 at their cell surface, etc. As a consequence, a PBMC population may be detected through the use of an antibody specifically recognizing such a marker. A subpopulation of PBMC may express a given antibody specifically recognizing an antigen. As such, this subpopulation of PBMC is capable of specifically recognizing the antigen, in contrast to other PBMC that do not express the antibody.

The term “T cell” may refer to different T-cells that may include, but are not limited to, T-helper cells, cytotoxic T-cells, memory T-cells, regulatory T-cells (also known as suppressor cells), and natural killer T-cells. A T-cell can be distinguished from other lymphocytes by the presence of a T-cell receptor (TCR) on its cell surface. The TCR may include a CD3 complex. The CDR complex may be a group of cell surface molecules that associates with the TCR and functions in the cell surface expression of TCR and in the signaling transduction cascade that originates when a peptide: MEW ligand binds to the TCR. Accordingly, the term “CD3+ T-cell” is a T-cell that is associated with the CD3 complex. As discussed below, the biological sample may include at least one T-cell, and in some cases, the T-cell may be a CD3+ T-cell.

The term “antibody” may refer to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)2, and F(ab)2 fragments. An “antigenic determinant” is the portion of an antigen molecule that determines the specificity of the antigen-antibody reaction. An “epitope” refers to an immunological determinant of an antigen that serves as an antibody-binding site, such as the antigenic determinant of a polypeptide. In some embodiments, when the epitope is an amino acid sequence, an epitope may include as few as 3 amino acids in a spatial conformation which is unique to the epitope. Generally, an epitope may include at least 6 amino acids, and in some cases, at least 8-10 amino acids. Many antibodies are available commercially and in addition, antibodies also may be produced by methods well known in the art, e.g., by immunizing animals with the antigens. Antigens may be isolated from samples based on their binding characteristics. Alternatively, if the antigen is a protein and the amino acid sequence is known, the protein may be synthesized and used to generate antibodies by methods well-known in the art.

The term “sample,” “patient sample,” “biological sample,” and the like, may encompass a variety of sample types obtained from a patient, individual, or subject and may be used in a diagnostic or monitoring assay. The sample may be obtained from a healthy subject, a diseased patient, or a patient exposed to a virus.

The term “control sample” may mean any control or standard familiar to one of ordinary skill in the art useful for comparison purposes. For example, the control sample may be one that contains no antigen.

EBV is a virus that contains a single-stranded RNA genome that encodes seven viral proteins: nucleoprotein (NP), glycoprotein (GP), polymerase (L), VP24, VP30, VP35, and VP40. EBV is configured to invade almost all human cells using different attachment mechanisms for each cell type, except for lymphocytes. In some cases, EBV enters target human cells by using different uptake mechanisms including lipid raft, receptor-mediated endocytosis, and micropinocytosis. An individual or subject infected with EBV may mount a strong inflammatory response. The inflammatory process is then followed by a T-cell response leading to the generation of EBV-specific IgG1 and IgG3 subclass responses and to marked and sustained activation of cytotoxic cells involved in the elimination of infected cells from peripheral circulation. In other words, EBV may infect the cells of a subject, which may display signals of an infection on the surface of the cell to activate T-lymphocytes—the white blood cells that are helpful in destroying other infected cells before the virus replicates further.

Different types of diagnostic testing are currently available to detect EBV, such as enzyme-linked immunosorbert assay (“ELISA”) and polymerase chain reaction (“PCR”). However, these known diagnostic tests are only capable of detecting the presence of virus specific markers after the onset of symptoms. Moreover, these types of tests generally have low sensitivity and may produce false negatives or false positives. Accordingly, a method and a diagnostic kit utilizing an enzyme-linked immunospot (“ELISpot”) assay enables the detection of EBV before the onset of symptoms due to the assay's sensitivity for detecting low frequencies of antigen reactive T-cells.

Previously, ELISPOT assay had only been established for diagnosis based on detectable responses of IFN-γ expressed by memory T-cells upon re-exposure to an antigen. Therefore, the ELISPOT assay's utility for pre-symptomatic applications had not been obvious as the development of antigen specific memory T-cells takes weeks or months after the initial infection. Rather, an ELISPOT assay demonstrated that T-cells were stimulated to secrete cytokines upon exposure to nucleoproteins (“NP”) and other EBV proteins. However, EBV specific T-cells appear to be present in infected PBMC samples prior to symptoms, since T-effector cells usually start differentiating from the precursor Naïve cells as early as 24 hours after exposure (Wherry & Ahmed 2004, Jour. of Virology 78: 5535-45). Furthermore, T-effector cells lyse infected host cells after antigenic re-stimulation, and both T-effector and T-memory cells can be stimulated to generate a detectable level of interferon (Todryk 2009 Immunology, 128, 83-91). Therefore, based on the interrogation of EBV specific T-effector cells at the earliest stage of infection (if present in the PBMC/blood samples), the stimulation of EBV specific T-effector cells with Ebola-virus like particles (“VLP”) antigens may generate a detectable interferon gamma (“IFN-γ”) response. The VLP contains all or most of the available EBV antigenic epitopes and ensures to capture a wide range of antigens that may elicit immune response at the earliest stage of infection.

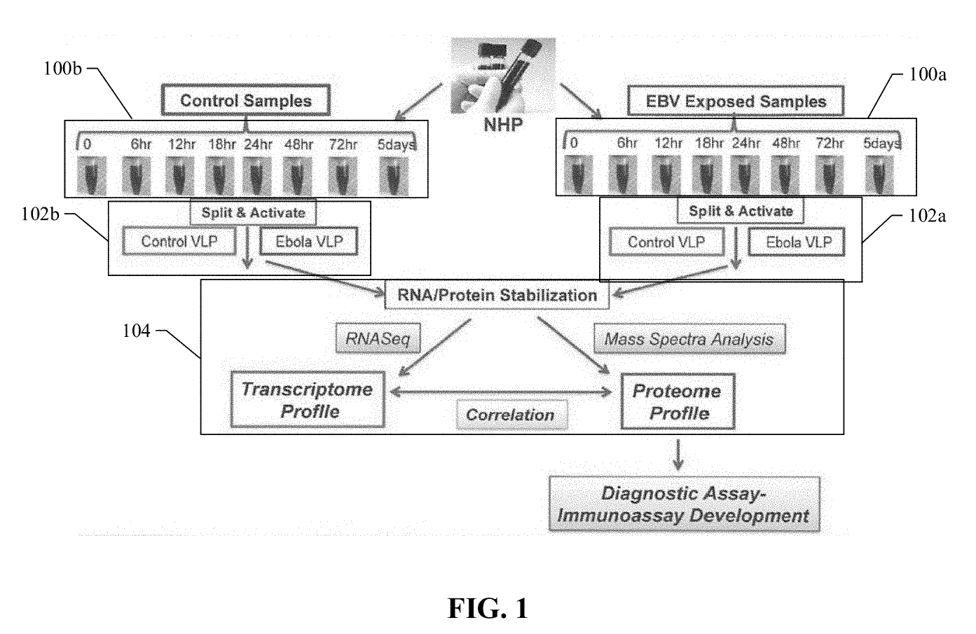

Accordingly, a method for the pre-symptomatic diagnosis of a viral illness, such as EBV, in a subject is provided. FIG. 1 illustrates a method for the pre-symptomatic diagnosis of the viral illness according to an example embodiment. As shown in FIG. 1, the method may include, at step 100a, a health care professional or clinician obtaining a biological sample from a subject. In some cases, the subject may suspect that they may have been exposed to the virus. Furthermore, the biological sample may be obtained by the health care professional by drawing blood from the subject. It should be understood that the subject may be a human subject. In some cases, however, the subject may be a non-human primate such as a monkey, ape, or the like. Accordingly, the method may be used for medical, veterinary, or development purposes. Furthermore, as shown in step 100a, a time range in which the biological sample may be obtained by the health care profession may vary based on the route of exposure, type of animal being tested, number of EBV particles, etc. For example, the time range in which the biological sample may be obtained may vary from 0 hours to five days within suspected exposure to the virus, or in some cases later than five days. In accordance with some example embodiments, a sample from a non-human primate having 1,000 PFU/μ of EBV demonstrates that the non-human primate develops symptoms at day five. Accordingly, the sample may be obtained prior to day five. It should be understood that at low doses or in other routes of exposure the presentation of symptoms may be delayed in the subject.

In accordance with example embodiments, the biological sample may include at least one PBMC from a subject. In some cases, the PBMC may include at least one T-cell. In further example embodiments, the T cell may be a CD3+ T cell. After obtaining the biological sample, at step 102a, the at least one PBMC may be isolated or separated from the overall biological sample. The isolation of the at least one PBMC may be done by any method known to one skilled in the art. For example, in some cases, the isolation of the at least one PBMC from the overall biological sample may be done by density gradient centrifugation.

As further shown in step 102a in FIG. 1, upon the isolation of the at least one PBMC from the biological sample, the at least one PBMC may then be stimulated or activated. The stimulation of the at least one PBMC may include adding a predetermined amount of an antigen associated with the viral illness to trigger an IFN-γ response by the T-cells in the at least one PBMC sample and adding a predetermined amount of reagent configured to capture the T-cell IFN-γ response. In some cases, a T-cell IFN-γ response means that the T-cell or CD3+ T-cell is responding to an environmental stimulus, such as the presence of an antigen, by producing IFN-γ. In other words, if a T-cell IFN-γ response is detected, then the subject has been previously exposed to, or encountered previously, the viral illness. A subject that has not had prior exposure to the viral illness will not respond to the antigen from the viral illness by producing IFN-γ (i.e., the subject does not have the viral illness). In some cases, peptide antigens may achieve detectable stimulation of the at least one PBMC at 1 mg (100 L/well of 10 g/mL sample). Further, ELISPOT counts (based on IFN-γ detection) are linear in the range between 2.5×10⁴ and 10⁶ of PBMCs.

The step of adding the predetermined amount of reagent may include pre-coating a well configured to be used in the ELISpot assay with a predetermined amount of reagent. It should be understood that the reagent used to capture IFN-γ may vary based on the T-cell response (i.e., amount added, etc.). However, the amount of reagent used per well may be in the range of 200 ng to 1 g. In some cases, the reagent may be an antibody. Thus, in some case, the antibody may be a polypeptide or group of polypeptides which may include at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)2, and F(ab)2 fragments. In other words, the ELISPOT assay may be used to measure the numbers of T-cells that secreted IFN-γ upon stimulation by the antigen associated or correlated with a virus (e.g., EBV).

Once the well is pre-coated with the antibody, the at least one PBMC may be added to the well. Then, the stimulation of the at least one PBMC may further include adding to or mixing the at least one PBMC with at least one antigen from the virus or viruses that the subject may have been exposed to. In some cases, for example, the at least one PBMC may be added to or mixed with only one antigen. In other example embodiments, however, the PBMC may be added to or mixed with a plurality of antigens if the subject suspects they may have exposed to multiple viruses or is unsure of the virus the subject has been exposed to. Accordingly, the method may be beneficial in limiting the number of tests that a health care professional needs to run on the subject's biological sample if the subject is unsure what viruses they have been exposed to. In other words, rather than a health care professional having to run multiple tests, the at least one PBMC may be exposed to multiple antigens at the same time to see which virus, out of a plurality of viruses, that the subject may have contracted.

It should be understood that the antigen or antigens added to or mixed with the at least one PBMC may be immunodominant or recognized significantly by T-cells after being exposed to viral infection. Furthermore, as mentioned above, antigens used in the method and diagnostic kit described herein may include polypeptides, peptides, polysaccharides, or the like. In some embodiments, the antigen may include at least one protein or fragment thereof, such as the immunogenic part of the protein that is recognized by the T-cell. Moreover, the at least one protein or fragment may be at least one purified protein or fragment thereof. In further embodiments, the at least one protein or fragment may be at least one recombinant protein or fragment thereof. The proteins may be used to test the at least one PBMC for the ability to secrete IFN-γ.

In accordance with an example embodiment, the antigen may be added to the at least one PBMC in the form of an extract or lysate. The extract or lysate may be a solution of cellular proteins resulting when cells are lysed or broken apart, such as by shearing cells. In some cases the extract or lysate may be in crude, partially purified, or purified form. A crude extract or lysate may be solution formed when cells are lysed or broken apart with only minimal purification of the cell antigens away from remaining cell components. A partially purified extract or lysate may be solution in which antigens have been extracted or purified away from some of the remaining components of the cell. A purified extract or lysate may be a solution in which antigens have been completely or substantially separated from remaining components of the cell. Accordingly, in some embodiments, the antigen used may be in a crude form when added to or mixed with the at least one PBMC. In further example embodiments, the antigen used may be in a partially-pure form when added to or mixed with the at least one PBMC. In even further example embodiments, the antigen used may be in a pure form when added to or mixed with the at least one PBMC. It should be understand that the extract or lysate may be purified by any method known to one skilled in the art. For example, the extract or lysate may be purified by the use of a detergent or a chaotropic agent to disrupt particles followed by differential extraction and separation of the polypeptides by ion exchange chromatography, affinity chromatography, sedimentation according to density, or gel electrophoresis.

After adding the antigen and the reagent to the at least one PBMC, the mixture of the antigen, reagent, and the at least one PBMC may then be incubated. In some cases, the mixture of the antigen, the reagent, and the at least one PBMC may be incubated for a predetermined time under a predefined condition. In some cases, the predetermined time and the predefined condition of the incubation may be dependent on the length of time since the subject's exposure to the viral illness. For example, the predefined time and condition used for incubation may be based on: i) subject's initial exposure date; ii) subject's duration or repeated contact with infectious individuals; iii) amount of infectious sample that the subject was exposed to (e.g., number of virus), route of exposure, etc.

In some cases, the mixture may be incubated anywhere from 1-5 hours at room temperature. For example, if the subject suspects they were exposed recently to the viral illness, such as in the last 1-2 days, the incubation time period for the mixture may be longer, such as 3-5 hours. However, if the subject suspects they were exposed to the viral illness within the last 3-5 days or longer, the incubation time period for the mixture may only be 1-3 hours.

After the incubation period, the mixture of the at least one PBMC, antigen, and reagent may then be analyzed as shown in step 104. The analysis of the mixture may include the analysis of at least two profiles: (1) analysis and measurement of transcriptomic profiles through techniques such as microarray, RNA-seq, etc., and (2) analysis and measurement of proteomic profiles through techniques such as gel electrophoresis and mass spectrometry. The transcriptomic and proteomic profiles of infected and control samples after stimulation with VLPs may provide additional predictive immune markers (other than INT-γ) that could be utilized to assess pre-symptomatic exposure status. Accordingly, the analysis of these profiles may include detecting and measuring the IFN-y response of the T-cell, if any.

Furthermore, the method may include comparing the levels of IFN-y detected in the mixture of the PBMC, reagent, and antigen to the levels of IFN-y in a control sample. In some cases, the control sample may be a biological sample from the subject that contains no antigen. Accordingly, in order to ensure an effective comparison of the antigen-treated mixture to the control sample, the biological sample for use as the control sample may be treated similarly to the antigen-treated mixture. In other words, as shown in step 100b, the healthcare professional may obtain a second biological sample that contains at least one peripheral blood mononuclear cell from the subject for use as the control sample. It should be understood that the control sample is a biological sample from the subject where at least one PBMC may be isolated from the overall biological sample. As shown in step 102b of FIG. 1, upon the isolation of the at least one PBMC for use as the control sample, the at least one PBMC may then be mixed with the reagent (e.g., in a pre-coated well). However, no antigen will be added to or mixed with the at least one PBMC. Thus, when detecting or determining the IFN-y levels in the control sample, the IFN-y level may be at or approximately zero.

Accordingly, the comparison of the levels of IFN-γ in the antigen-treated mixture to the levels of IFN-γ in the control sample may include assessing the proportion or level of IFN-γ, or number of IFN-γ producing cells in antigen-treated mixture to the proportion or level of IFN-γ or number of IFN-γ producing cells in the mixture that has not been treated with antigen. If the difference between the antigen-treated mixture and the control sample is greater than a predetermined amount, the subject may have been infected with the viral illness. In some cases, the predetermined amount may be greater than three to five times above the standard deviation. It should be understood, however, that the if there is a high level of basal background, the predetermined amount may be greater than ten times above the standard deviation.

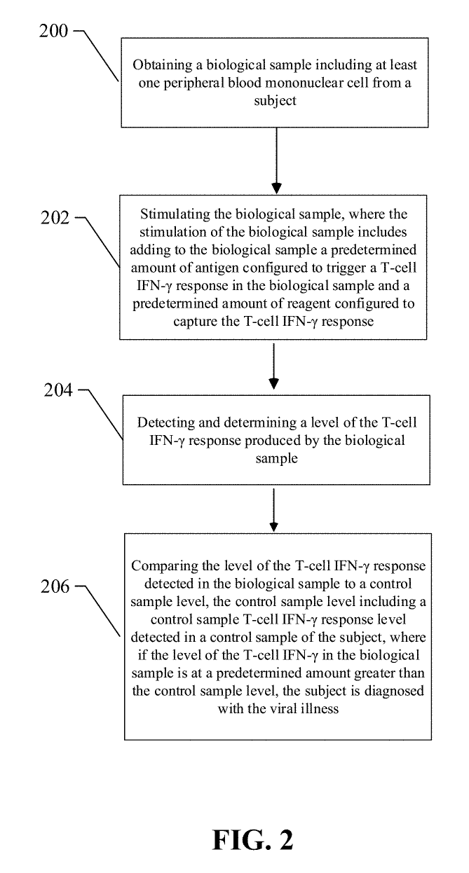

Furthermore, FIG. 2 illustrates a flow diagram for the method associated with the pre-symptomatic diagnosis of a viral illness. As shown in FIG. 2, the method comprises an initial step 200, in which biological sample from the subject is obtained, the biological sample including least one PBMC from the subject. Next, in step 202, the biological sample is stimulated. As discussed above, the stimulation of the biological sample may include adding a predetermined amount of antigen configured to trigger a T-cell IFN-γ response in the biological sample and a predetermined amount of reagent configured to capture the T-cell IFN-γ response. In step 204, the detection and determination of the level of the T-cell IFN-γ response produced by the biological sample occurs. Finally, in step 206, the level of the T-cell IFN-γ response detected in the biological sample is compared to a control sample level, the control sample level including the T-cell IFN-γ response in the control sample of the subject. If the level of the T-cell IFN-γ in the biological sample is at a predetermined amount greater than the control sample level, the subject is diagnosed with the viral illness.

Accordingly, the method may include the use of a mixture of an antigen such as viral peptides or full proteins or VLP to contact with blood or PBMC samples. The assay format may be ELISPOT or other immunoassay format such as ELISA that assesses the differential expression of immune response markers (e.g., IFN-γ) compared to the control samples. The assay may be a transcriptomics or proteomics-based analysis that assesses the differential expression of immune response markers (e.g., IFN-γ) compared to the control samples at the RNA or protein levels, respectively. In some cases, stimulation values (i.e., those IFN-γ levels or values detected in the antigen-treated sample) may be compared to the same individual's sample that was drawn before entering an pandemic zone, or prior to having contact with an infectious individual (i.e., the control sample).

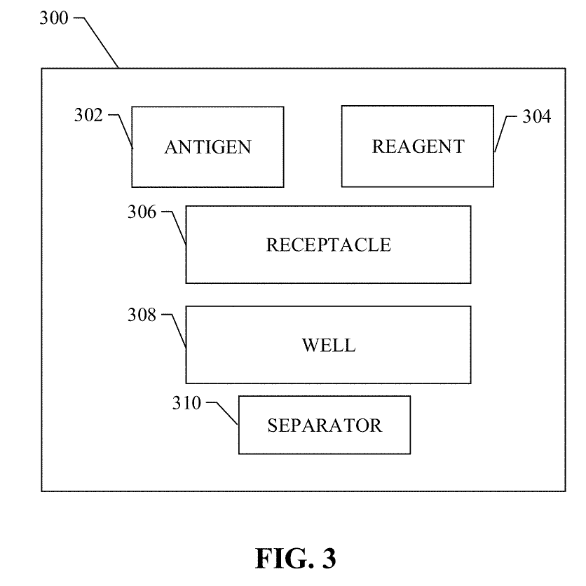

In a further example embodiment, a diagnostic kit for practicing the above-disclosed method may be provided. FIG. 3 illustrates the diagnostic kit for the pre-symptomatic diagnosis of a viral illness according to an example embodiment. In general, the diagnostic kit 300 may contain some or all of the components, reagents, supplies, or the like to practice the above-disclosed method. Accordingly, the diagnostic kit 300 may refer to any intended article of manufacture (e.g., a package or a container) that includes an antigen, reagent, and a set of particular instructions for practicing the method.

Thus, the diagnostic kit 300 for the pre-symptomatic diagnosis of the viral infection may include at least one antigen 302 from the viral illness that the subject suspects they were exposed to and an reagent 304 for capturing the IFN-γ produced by the biological sample from the subject.

In further example embodiments, the diagnostic kit 300 may also include a receptacle 306 for collecting the biological sample from the subject, such as a syringe, tube, a blood collection tube, or the like. In further embodiments, the diagnostic kit may include a well 308 for conducting the ELISpot assay. In some cases, the well may be pre-coated with the reagent 304. Furthermore, the diagnostic kit 300 may also include a separator 310 for separating the at least one PBMC from the other components of the biological sample of the subject.

Example embodiments therefore represent a method and a diagnostic kit for the pre-symptomatic diagnosis of a viral illness. The method may include obtaining a biological sample comprising at least one peripheral blood mononuclear cell from a subject. The method may further include stimulating the biological sample, where the stimulation of the biological sample includes adding to the biological sample a predetermined amount of antigen configured to trigger a T-cell IFN-γ response in the biological sample and a predetermined amount of reagent configured to capture the T-cell IFN-γ response. The method may even further include comparing the level of the T-cell IFN-γ response detected in the biological sample to a control sample level, the control sample level including a control sample T-cell IFN-γ response level detected in a control sample of the subject, where if the level of the T-cell IFN-γ response in the biological sample is at a predetermined amount greater than the control sample level, the subject is diagnosed with the viral illness.

In some embodiments, additional optional steps and/or features may be included or the steps/features described above may be modified or augmented. Each of the additional features, steps, modifications, or augmentations may be practiced in combination with the steps/features above and/or in combination with each other. Thus, some, all or none of the additional features, steps, modifications, or augmentations may be utilized in some embodiments. Some example additional optional features, steps, modifications, or augmentations are described below, and may include, for example, detecting the level of the T-cell IFN-γ response produced by the biological sample using an enzyme-linked immunosorbent spot (ELISPOT) assay. Alternatively or additionally, the reagent configured to capture the T-cell IFN-γ response may be an antibody. Alternatively or additionally, obtaining the biological sample may include isolating the at least one PBMC from other components of the biological sample. Alternatively or additionally, the viral illness may be Ebola Virus Disease, and the antigen may be an Ebola Virus Disease antigen. Alternatively or additionally, the at least one PBMC may include at least one CD3+ T cell. Alternatively or additionally, the predetermined amount greater than the control sample level may greater than 2.5 times above a standard deviation. Alternatively or additionally, the predetermined amount greater than the control sample level may be greater than 5 times above the standard deviation. Alternatively or additionally, the stimulation of the biological sample may include incubating the biological sample, the reagent, and the antigen for a predetermined time. Alternatively or additionally, the predetermined time for incubating the biological sample may be for a period of 1-5 hours. Alternatively or additionally, the predetermined time for incubating the biological sample may be for a period of 1-2 hours. Alternatively or additionally, the predetermined amount of antigen may be 0.1 ng to 10,000 ug. Alternatively or additionally, obtaining the biological sample may include obtaining the biological sample within five days from suspected exposure to the viral illness.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

What is claimed is:

1. A method for a pre-symptomatic diagnosis of a viral illness in a subject, the method comprising:
(a) obtaining a biological sample comprising at least one peripheral blood mononuclear cell from a subject;
(b) stimulating the biological sample, wherein the stimulation of the biological sample comprises adding to the biological sample a predetermined amount of antigen configured to trigger a T-cell IFN-γ response in the biological sample and a predetermined amount of reagent configured to capture the T-cell IFN-γ response;
(c) detecting and determining a level of the T-cell IFN-γ response produced by the biological sample; and
(d) comparing the level of the T-cell IFN-γ response detected in the biological sample to a control sample level, the control sample level comprising a control sample T-cell IFN-γ response level detected in a control sample of the subject, wherein if the level of the T-cell IFN-γ response in the biological sample is at a predetermined amount greater than the control sample level, the subject is diagnosed with the viral illness.

2. The method of claim 1, wherein the detecting of the level of the T-cell IFN-γ response produced by the biological sample comprises detecting the level of the T-cell IFN-γ response produced by the biological sample using an enzyme-linked immunosorbent spot (ELISPOT) assay.

3. The method of claim 1, wherein the reagent configured to capture the T-cell IFN-γ response is an antibody.

4. The method of claim 1, wherein obtaining the biological sample comprises isolating the at least one PBMC from other components of the biological sample.

5. The method of claim 1, wherein the viral illness is Ebola Virus Disease, and wherein the antigen is an Ebola Virus Disease antigen.

6. The method of claim 1, wherein the at least one PBMC comprises at least one CD3+ T cell.

7. The method of claim 1, wherein the predetermined amount greater than the control sample level is greater than 2.5 times above a standard deviation.

8. The method of claim 7, wherein the predetermined amount greater than the control sample level is greater than 5 times above the standard deviation.

9. The method of claim 1, wherein the stimulation of the biological sample further comprises incubating the biological sample, the reagent, and the antigen for a predetermined time.

10. The method of claim 9, wherein the predetermined time for incubating the biological sample is for a period of 1-5 hours.

11. The method of claim 10, wherein the predetermined time for incubating the biological sample is for a period of 1-2 hours.

12. The method of claim 1, wherein the predetermined amount of antigen is 0.1 ng to 10,000 ug.

13. The method of claim 1, wherein obtaining the biological sample further comprises obtaining the biological sample within five days from suspected exposure to the viral illness.

14. A diagnostic kit for a pre-symptomatic diagnosis of a viral illness in a subject, the diagnostic kit comprising:
a) a predetermined amount of antigen associated with the viral illness; and
b) a predetermined amount of reagent for capturing IFN-γ produced by a biological sample from the subject.

15. The diagnostic kit of claim 14, wherein the diagnostic kit further comprises a well, the well being pre-coated with the predetermined amount of reagent for capturing the IFN-γ produced by the biological sample from the subject.

16. The diagnostic kit of claim 14, wherein the reagent configured for capturing the T-cell IFN-γ response is an antibody.

17. The diagnostic kit of claim 14, wherein the predetermined amount of antigen is 0.1 ng to 10,000 ug.

18. The diagnostic kit of claim 14, wherein the biological sample from the subject is obtained from the subject within five days of suspected exposure to the viral illness.

19. The diagnostic kit of claim 14, wherein the viral illness is Ebola Virus Disease, and wherein the antigen is an Ebola Virus Disease antigen.

20. The diagnostic kit of claim 14, wherein the diagnostic kit further comprises a receptacle for collecting the biological sample from the subject.
